(19) United States
(12) Patent Application Publication
(10) Pub. No.: US 2006/0007395 A1
(43) Pub. Date: Jan. 12, 2006

(54) System and method for wavefront measurement

(76) Inventors: William T. Mayo, Seal Beach, CA (US); Anthony Y. Van Heugten, Sarasota, FL (US)

Correspondence Address: PERKINS COIE LLP, POST OFFICE BOX 1208, SEATTLE, WA, 98111-1208, US

(21) Appl. No.: 10/885,504

(22) Filed: Jul. 6, 2004

(51) Int. Cl. A61B 3/10; A61B 3/00

(52) U.S. Cl. 351/205; 351/246

(57) ABSTRACT

A wavefront measuring system and method for detecting aberrations in wavefronts that are reflected from, transmitted through, or internally reflected within objects sought to be measured, e.g., optics systems such as the human eye. The system includes one or more reticles in the path of a return wavefront from the object, and a detector at a diffraction pattern self-imaging plane relative to the reticle(s). A diffraction pattern of the wavefront is analyzed and results in a model of the wavefront phase characteristics. A set of known polynomials may be fitted to the wavefront phase gradient to obtain polynomial coefficients that describe aberrations in the object, or within the wavefront source being measured.

### BACKGROUND

Improving eyesight is vitally important. Precise measurement of the eye's physical characteristics, including features of the eye, is necessary to accurately prescribe vision correction. With the advent of technologies capable of creating highly complex optical surfaces, a resurgence of interest has arisen in the tools required to measure the eye's optical characteristics to a higher degree of complexity than was previously possible. In particular, wavefront measurement systems have been developed to measure the physical characteristics of the eye.

In a typical wavefront measurement system, a light beam is projected into the eye, which focuses the light beam onto the retina of the eye. The light beam then reflects back out of the eye through the optical components of the eye. A relay lens system typically collects the light reflected from the eye, and projects the collected light through one or more reticles. The light that passes through the reticle(s) is projected onto a translucent screen to create an image on the screen. A charged coupled device (CCD) camera, or similar device, is focused onto the screen to “see” the shadow patterns created by the reticle(s), and the shadow pattern data is imaged onto a CCD chip. A computer or other processor converts the CCD camera images into digital data. The computer then analyzes the data to determine the refractive condition of the eye.

In such a system, the information contained within the shadow patterns is generally somewhat degraded because the light is passed onto a screen, and through the camera's focus optics, before being imaged onto the CCD chip. Additionally, the system requires significant optical length to allow space for the imaging screen and the focus optics of the camera. Accordingly, a need exists for an improved wavefront measuring system that exhibits reduced image degradation, requires less optical length, and/or is less costly than existing measurement systems.

### SUMMARY OF THE INVENTION

The invention is directed to systems and methods for determining aberrations in, or the shape of, a wavefront (i.e., a coherent electromagnetic wavefront). The system includes one or more reticles that are positioned in the path of the wavefront, and a light detector positioned in the wavefront path downstream from the reticles. The light detector is located at a diffraction pattern self-imaging plane, commonly referred to as a Talbot plane, relative to the reticle. Shadow patterns of the wavefront that are produced by the reticle(s) are imaged onto the light detector. A computer or other processor receives an output signal from the light detector identifying the shadow patterns. The computer then analyzes the shadow patterns to calculate aberrations in the wavefront.

The light detector may be a CCD camera, or any other suitable electronic camera or other light-detecting device. By locating the light detector at a diffraction pattern self-imaging plane relative to the reticle, i.e., directly at the plane where shadow patterns form, the shadow patterns can be imaged directly onto the light detector.

Other features and advantages of the invention will appear hereinafter. The features of the invention described above can be used separately or together, or in various combinations of one or more of them. The invention resides as well in sub-combinations of the features described.

### BRIEF DESCRIPTION OF THE DRAWINGS

In the drawings, wherein similar reference characters denote similar elements throughout the several views:

FIG. 1 is a schematic diagram of a preferred wavefront measuring system using one reticle.

FIG. 2 is a schematic diagram of a preferred wavefront measuring system using two reticles.

### DETAILED DESCRIPTION OF THE DRAWINGS

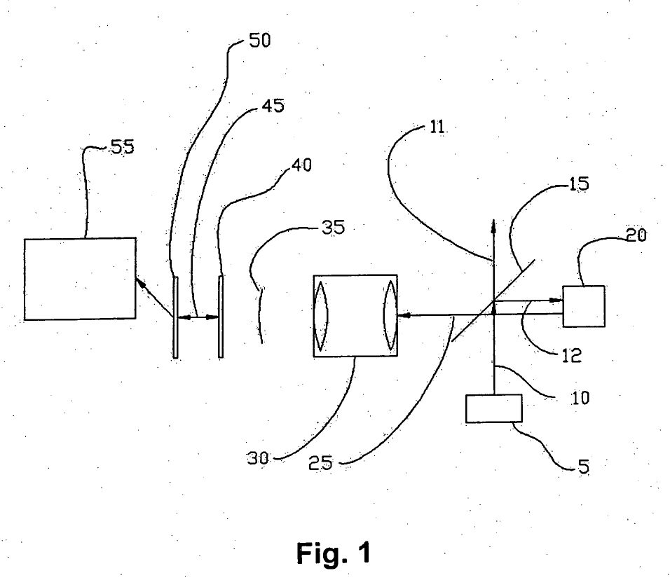

FIG. 1 illustrates a wavefront measuring system according to a first preferred embodiment. The wavefront measuring system includes a light source 5 for creating a source wavefront 10. The light source 5 is preferably a laser source, a light-emitting diode, or any other suitable light-generating device. The wavelength spectrum of the wavefront produced preferably has a narrow width, for example, approximately 20 nanometers, but the system could function with a broader spectrum. The wavefront preferably has a wavelength of approximately 780 nanometers. Higher wavelengths, which are less representative of an eye's true refractive index, and lower wavelengths, which may cause a patient's pupil to constrict upon exposure to the light, may alternatively be used.

A beam splitter 15 for reflecting a first portion 12 of the source wavefront 10 toward an object 20 to be measured is located downstream from the light source 5. A second portion 11 of the source wavefront 10 generally passes through the beam splitter 15 as loss. The object 20 to be measured may be any object with refractive properties, and is preferably a human eye.

The first portion 12 of the wavefront reflects from the object 20 and passes through the beam splitter 15, as a third wavefront portion 25, toward a relay optics system 30 or a similar device. The third wavefront portion 25 is projected, as a wavefront to be measured 35, by the relay optics system 30 toward a reticle 40 having one or more grating lines. The wavefront to be measured 35 passes through the reticle 40, and travels a specific path distance 45 to a light detector 50. The specific path distance 45 represents the longitudinal distance from the reticle 40 to a diffraction pattern self-imaging plane, or Talbot plane, relative to the reticle 40.

Determination of the specific path distance 45 is well known to those skilled in the art, and is described in detail in “Fourier Transform Method For Automatic Processing of Moiré Deflectograms,” Quiroga et al., Opt. Eng. 38(6) pp. 974-982 (June 1999), and “Refractive Power Mapping of Progressive Power Lenses Using Talbot Interferometry and Digital Image Processing,” Nakano et al., Opt. Laser Technology. 22(3), pp. 195-198 (1990), which are hereby incorporated by reference.

In general, the location of a Talbot plane is dependent upon the wavelength of the wavefront being measured and the spacing between grating lines in a reticle, and can be readily calculated by those skilled in the art. The location of a Talbot plane, i.e., of the diffraction pattern self-imaging plane, is generally at a longitudinal distance of approximately
d=(np2/λ)
from a reticle, where p is the grating spacing of the reticle, λ is the spectral wavelength of the wavefront, and n is an integer representing the desired Talbot plane (for example, if n=2, then d represents the longitudinal distance from the reticle to the second Talbot plane).

A self-image of the wavefront is formed on the light detector 50, preferably in the form of shadow patterns. The light detector is preferably a CCD camera chip or other suitable detecting device. The light detector 50 outputs the self-image of the shadow patterns to a computer 55 or other processor, which preferably digitizes the shadow patterns. The computer 55 analyzes the shadow patterns to extract refractive information pertaining to the object 20. Mathematical processes for extracting the refractive information from the shadow patterns are well known to those skilled in the art, and are described in detail in, for example, the incorporated Nakano et al. reference.

One basic method for extracting the refractive information involves examining a shadow pattern of a known reference object, and comparing it to the shadow pattern of the object 20 to be measured. The displacement of the shadow patterns between the reference object and the object 20 to be measured, both in terms of direction and magnitude, indicates the refractive properties of the object 20.

By locating the light detector 50 at a diffraction pattern self-imaging plane, or Talbot plane, relative to the reticle 40, i.e., directly at the plane where the shadow patterns form, the shadow patterns can be imaged directly onto the light detector 50. As a result, the need for a translucent imaging screen and camera focus optics, which could otherwise degrade information contained in the shadow patterns, is eliminated. Accordingly, the wavefront measurement system exhibits less image degradation, and requires less space and fewer costly optical components, than existing systems using those components.

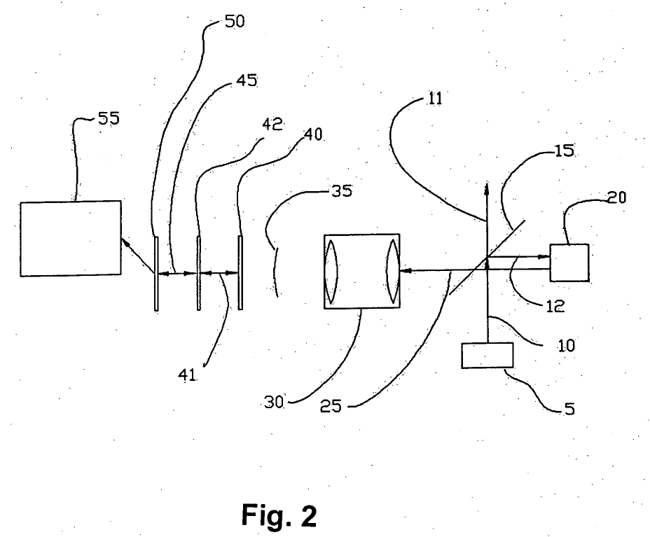

FIG. 2 illustrates an alternative preferred embodiment of a wavefront measuring system. In this embodiment, the system's operation is the same as described in FIG. 1, up to the point where the wavefront to be measured 35 passes through the reticle 40. Instead of self-imaging on the light detector 50, the wavefront 35 self-images onto a second reticle 42, which is preferably rotated slightly with respect to the initial reticle 40, creating a moiré effect. Moiré effects are well known to those skilled in the art, and are described in detail in the incorporated Quiroga et al. reference.

A second self-imaging occurs at the plane where the light detector 50 is located, and the shadow patterns of the wavefront 35 are imaged directly onto the light detector 50. An advantage of adding a second reticle 42 is that the moiré effect created by changing the rotation between the two reticles 40, 42 can control the density of fringe shadow patterns. As a result, the amount of movement of the shadow patterns per wave of change in the wavefront being measured is increased, thus making the system more responsive.

The distance 41 between the two reticles 40, 42 preferably corresponds to a Talbot plane location, i.e., the second reticle 42 is preferably located at the first Talbot plane, or the second Talbot plane, etc. relative to the first reticle 40. Any deviation from this Talbot plane spacing will result in a diminishing of intensity of the shadow patterns produced. Additional reticles may be used to add further control to the resolution and response of the movement of the shadow patterns. Each reticle is preferably located at a Talbot plane relative to the previous reticle, and the light detector 50 is preferably located at a Talbot plane relative to the last reticle, so that the detector 50 receives and detects shadow patterns from the last reticle.

In a preferred embodiment, a computer program quantifies how much the wavefront deviates from perfectly flat, or in other words, from the phase gradient of the wavefront phase-front. The deviations are expressed mathematically, preferably as polynomials, and are generally quantified in terms of the numbers of waves of light, or fractions of waves of light, by which the wavefront deviates from flat at any given location in the wavefront.

The wavefront information is preferably obtained by performing a Fourier Transform on the wavefront, or in other words, a transformation of the wavefront from the spatial image domain into the spatial frequency domain. While performing this step, the orientation of the reticle(s) must be taken into account. In a preferred method, the wavefront is analyzed in at least two directions (derivatives of specific phases of the wavefront may be determined), generally across the wavefront's horizontal and vertical axes, and the results are categorized into predefined aberrations known as “Zernike polynomials.” Zernike polynomials are commonly used to express wavefront measurements, and have been proposed as the ANSI standard in this regard.

In one method, coefficients representative of aberrations in the wavefront are determined by fitting derivative functions of a set of known polynomials to the measured deviations in the wavefront. In some instances, only selected portions of the wavefront in the spatial frequency domain are used to determine the coefficients. Once directional derivatives associated with a light beam that has passed through a reticle are determined, the derivatives can be used to output a measure of aberrations in the light beam.

One method for measuring wavefront deviation from flat includes passing a wavefront through an object, or reflecting it off of an object, then passing the wavefront through one or more reticles. Derivatives are then calculated as described above, and are used to describe the wavefront shape, as it deviates from flat. The aberrations in the wavefront are created by the optical components through which the wavefront passes.

The computer program analyzes the images produced by the wavefront, or a frequency transformation of the wavefront, as it passed through the reticle(s). The computer program converts these images into digital signals that are preferably stored in the computer's memory. The computer then executes the program to report the aberrations in the wavefront being analyzed. The output can be expressed in many ways, one of which is to find a best fit With the Zernike polynomials.

One or more filtering devices, such as a computational matte screen, may be used to remove unwanted noise from signals in the system, as is known in the art. Corrective optics, such as contact lenses or eyeglasses, may be designed based on the measured aberrations, to correct a patient's vision.

While embodiments and applications of the present invention have been shown and described, it will be apparent to one skilled in the art that other modifications are possible without departing from the inventive concepts herein. The invention, therefore, is not to be restricted except by the following claims and their equivalents.

What is claimed is:

1. A system for determining aberrations in an electromagnetic wavefront, comprising:
at least one source of the electromagnetic wavefront directing a beam onto an object system, the object system reflecting or passing at least part of the beam to render a wavefront to be analyzed;
at least one reticle positioned in a path of the wavefront to be analyzed;
at least one detector positioned to detect the wavefront passing through the reticle, the detector being located at a diffraction pattern self-imaging plane relative to the reticle; and
at least one processor receiving an output signal from the light detector and determining at least one aberration in the wavefront based thereon, the aberration representing at least one aberration in the object system.

2. The system of claim 1, wherein the processor executes logic to undertake method acts comprising:
accessing mathematical functions to characterize the electromagnetic wavefront; and
determining directional derivatives of the electromagnetic wavefront using the mathematical functions.

3. The system of claim 2, wherein the method acts include determining coefficients of polynomials based on at least one gradient of a phase-front of the wavefront, the coefficients being representative of aberrations.

4. The system of claim 3, wherein the method acts further include transforming the wavefront from a spatial image domain into a spatial frequency domain, prior to the act of determining coefficients.

5. The system of claim 4, wherein the act of determining coefficients includes determining directional derivatives of phases of the wavefront.

6. The system of claim 5, wherein directional derivatives are determined in at least two directions.

7. The system of claim 6, wherein the coefficients are determined by fitting derivative functions of a set of known polynomials to the derivatives obtained during the determining act.

8. A method for determining aberrations in an object system, comprising: passing a light beam from the object system through a reticle;
determining directional derivatives associated with the light beam subsequent to the light beam passing through the reticle; and
using the derivatives to output a measure of aberrations in the light beam.

9. The method of claim 8, further comprising transforming a wavefront associated with the light beam from a spatial image domain into a spatial frequency domain.

10. The method of claim 9, further comprising determining coefficients of polynomials based on the directional derivatives.

11. The method of claim 10, wherein the act of determining derivatives includes determining derivatives of phases of the wavefront.

12. The method of claim 11, comprising determining directional derivatives in at least two directions.

13. The method of claim 12, wherein the coefficients are determined by fitting derivatives of a set of known polynomials to data obtained during the determining act.

14. The method of claim 8, comprising locating a light detector at a diffraction pattern self-imaging plane relative to the reticle, to detect the wavefront.

15. A computer program product, comprising:
a computer readable medium having a program of instructions stored thereon for causing a digital processing apparatus to execute method steps for determining aberrations in at least one object, comprising:
means for receiving at least one representation of a wavefront propagating from the object;
means for determining directional derivatives of the representation;
means for fitting the directional derivatives to known polynomials or derivatives thereof to obtain coefficients of polynomials; and
means for outputting at least one signal based at least in part on the coefficients, the signal representing aberrations in the object.

16. The program product of claim 15, further comprising means for generating a frequency domain representation of the wavefront.

17. The program product of claim 16, wherein the means for determining determines derivatives of phases in two directions.

18. An apparatus for detecting aberrations in an object system as manifested in a wavefront from the object system, comprising:
at least one reticle positioned in a path of the wavefront;
at least one light detector positioned relative to the reticle to receive a self-image of at least one diffraction caused pattern associated with the wavefront; and
at least one processor receiving signals from the light detector representative of the self-image and deriving derivatives associated therewith, the processor using the derivatives to determine the aberrations.

19. The apparatus of claim 18, wherein the processor receives a frequency transformation of the wavefront and derives derivatives associated with phases of the frequency transformation.

20. The apparatus of claim 19, wherein the processor determines derivatives of phases in two directions.

21. The apparatus of claim 20, wherein the processor fits a set of known derivatives to the derivatives determined by the processor to obtain coefficients of polynomials representative of the aberrations.

22. A method for determining aberrations in a reflective or internally reflective object, comprising:
passing a light beam from the object through a reticle;
determining directional derivatives associated with the light beam subsequent to the light beam passing through the reticle; and
using the derivatives to output a measure of aberrations in the light beam and, hence, the object.

23. The method of claim 22, wherein the object is an eye of a patient.

24. The method of claim 23, further comprising transforming a wavefront associated with the light beam from a spatial image domain into a spatial frequency domain.

25. The method of claim 24, further comprising determining coefficients of polynomials based on the directional derivatives.

26. The method of claim 25, wherein the act of determining derivatives includes determining derivatives of phases of the wavefront.

27. The method of claim 26, comprising determining directional derivatives in at least two directions.

28. The method of claim 27, wherein the coefficients are determined by fitting derivatives of a set of known polynomials to data obtained during the determining act.

29. The method of claim 28, comprising locating a light detector at a diffraction, pattern self-imaging plane relative to the reticle; to detect the wavefront.

30. The system of claim 1, comprising a computationally implemented matte screen for removing unwanted noise from a signal.

31. The method of claim 8, comprising implementing a computational matte screen to filter a signal.

32. The system of claim 1, wherein the location of the self-imaging plane is a function of wavelength of the wavefront and spatial frequency of the reticle.

33. The apparatus of claim 18, wherein the location of the self-imaging plane is a function of wavelength of the wavefront and spatial frequency of the reticle.

34. The system of claim 4, wherein only selected portions in the spatial frequency domain are used to determine coefficients.

35. The apparatus of claim 21, wherein only selected portions in a spatial frequency domain are used to determine coefficients.

36. A system for determining the shape of an electromagnetic wavefront, comprising:
at least one reticle positioned in a path of the wavefront to be analyzed;
at least one detector positioned to detect the wavefront passing through the reticle, the detector being substantially located at a diffraction pattern self-imaging plane relative to the reticle; and
at least one processor receiving an output signal from the light detector and calculating the shape of the wavefront based thereon.

37. The system of claim 36, wherein the location of the self-imaging plane is a function of the wavelength of the wavefront and the spatial periodicity of the reticle.

38. The system of claim 36, wherein said reticle comprises a grating having a grating spacing, p.

39. The system of claim 37, wherein said diffraction pattern self-imaging plane is located in the near field a longitudinal distance of approximately
d=(np²/λ)
from said reticle, wherein p is the grating spacing of the reticle, A is the spectral wavelength of the wavefront, and n is an integer.

40. The system of claim 36, wherein said reticle comprises a grating having a grid-like pattern.

41. The system of claim 36, wherein the processor executes logic to undertake method acts comprising determining directional derivatives of the electromagnetic wavefront.

42. The system of claim 41, wherein the method acts further include transforming a diffraction pattern of the wavefront at the detector from a spatial image domain into a spatial frequency domain, prior to the act of determining coefficients.

43. The system of claim 42, wherein selected portions in the spatial frequency domain are used to determine said coefficients.

44. The system of claim 41, wherein the method acts include determining coefficients of polynomials based on at least one gradient of a phase-front of the wavefront, the coefficients being representative of the shape of the wavefront.

45. The system of claim 44, wherein the coefficients are determined by fitting derivative functions of a set of known polynomials to the derivatives obtained during the determining act.

46. The system of claim 41, wherein directional derivatives are determined in at least two directions.

47. The system of claim 41, wherein said method acts further comprise implementing a computational matte screen for filtering out noise.

48. A method for determining aberrations in an optical system comprising at least one optical element, said method comprising:
propagating a test beam along a path with said optical system in said path of said test beam so as to be illuminated by said test beam,
inserting a reticle in said path of said test beam at a location with respect to said optical system so as to receive light from said optical system, said light propagating through said reticle;
determining directional derivatives associated with said light subsequent to passing through the reticle; and
using the derivatives to output a measure of said aberrations.

49. The method of claim 48, further comprising transforming a diffraction pattern produced by said light passing through said reticle from a spatial image into a spatial frequency distribution.

50. The method of claim 48, further comprising determining coefficients of polynomials based on the directional derivatives.

51. The method of claim 50, wherein the coefficients are determined by fitting derivatives of a set of known polynomials to data obtained during the determining act.

52. The method of claim 48, comprising determining directional derivatives in at least two directions.

53. The method of claim 48, comprising locating a light detector at a position in said path so at to receive a self-image of the reticle.

54. The method of claim 48, further comprising implementing a computational matte screen as a filter.

55. A computer program product, comprising:
a computer readable medium having a program of instructions stored thereon for causing a digital processing apparatus to execute method steps for determining aberrations in a wavefront, comprising:
representing at least a portion of an image produced by said wavefront;
determining directional derivatives of the representation;
fitting the directional derivatives to known polynomials or derivatives thereof to obtain coefficients of polynomials; and
providing a wavefront characterization based at least in part on the coefficients, the wavefront characterization representing aberrations in the wavefront.

56. The program product of claim 55, further comprising generating a frequency domain representation of the wavefront.

57. The program product of claim 56, wherein the directional derivatives are determined in two directions.

58. An apparatus for characterizing an object with a wavefront from the object, comprising:
at least one reticle positioned in a path of the wavefront;
at least one light detector positioned relative to the reticle to receive a self-image diffraction pattern of the reticle produced by the wavefront; and
at least one processor receiving signals from the light detector representative of the self-image diffraction pattern and deriving derivatives associated therewith, the processor using the derivatives to characterize said object.

59. The apparatus of claim 58, wherein the object is an eye.

60. The apparatus of claim 58, wherein the location of the reticle is related to the wavelength of the wavefront and spatial frequency of the reticle.

61. The apparatus of claim 58, wherein the processor produces frequency transformation of the wavefront to produce a distribution in frequency space and derives derivatives of phases of the wavefront from the distribution in frequency space.

62. The apparatus of claim 58, wherein the processor determines derivatives of phases in two directions.

63. The apparatus of claim 58, wherein the processor fits a set of known derivatives to the derivatives determined by the processor to obtain coefficients of polynomials representative of the aberrations.

64. A method for determining aberrations in a reflective or internally reflective object system, comprising:
passing a light beam from the object system through a reticle, said light beam producing a near field diffraction pattern at said Talbot plane;
imaging said near field diffraction pattern at said Talbot plane;
using said near field diffraction pattern to output a measure of aberrations in the light beam.

65. The method of claim 64, wherein the object system is an eye and said method is for determining aberration in said eye.

66. The method of claim 64, further comprising transforming a wavefront associated with the light beam from a spatial image domain into a spatial frequency domain.

67. The method of claim 66, wherein only selected portions in said spatial frequency domain are used to determine coefficients.

68. The method of claim 64, comprising locating a light detector at said Talbot plane to detect the near field diffraction pattern.

69. The method of claim 64, further comprising designing corrective optics based on said measure of aberrations in said light beam so as to reduce said aberrations.

70. A system for measuring characteristics of the eye comprising:
means for generating an optical wavefront;
means for transmitting the optical wavefront to the eye, with the wavefront reflecting from a point on the retina of the eye;
means for transmitting the reflected wavefront from the eye through a reticle to create a shadow pattern;
a detector placed at a plane where the shadow pattern forms; and
means for analyzing the shadow pattern to produce measurement data relating to characteristics of the wavefront.
